(19) United States
(12) Patent Application Publication
(10) Pub. No.: US 2023/0316596 A1
(43) Pub. Date: Oct. 5, 2023

(54) Spatially Aware System and Method to Determine Perception of Supra-Threshold Visual Lightness Contrast with Adjustable Adapting Field, Associated Systems and Methods for Perceptually Uniform Calculation of Visual Contrast of Text and Non-Text Content, with Accommodation for Color Vision Deficiency.

(71) Applicant: Andrew Michael Somers, Los Angeles, CA (US)

(72) Inventor: Andrew Michael Somers, Los Angeles, CA (US)

(21) Appl. No.: 18/124,617

(22) Filed: Mar. 22, 2023

Related U.S. Application Data
(60) Provisional application No. 63/301,870, filed on Jan. 21, 2022.

(51) Int. Cl. G06T 11/00; G06T 7/90; A61B 3/02

(52) U.S. Cl. CPC G06T 11/001; G06T 7/90; A61B 3/022; G06T 2200/24; G06T 2207/10024; A61B 2503/12

(57) ABSTRACT

This invention provides a system and empirical study method to quantify perceived visual contrast, particularly at supra-threshold levels in conjunction with medium to high spatial frequency stimuli, such as text and text-like design elements. And further, to use the collected study data to instruct the implementation of systems and methods to calculate a perceived lightness contrast for a given stimulus and set of colors, and to do so in a perceptually uniform manner across the visual range of available colors on a self-illuminated display or device.

### CROSS-REFERENCE TO RELATED APPLICATIONS

### Claim for Priority

This patent application claims the priority date established by provisional application No. 63/301,870 filed 17:31:31 EST on 21 Jan. 2022 titled “Accessible, Perceptual, and Spatially Weighted Visual Contrast Algorithm with Methods for Color Vision Deficiency, Adaptation Adjustment, and Optimum Font Size and Weight Recommendation”. Relevant portions of said provisional application are hereby incorporated by reference.

### COPYRIGHT NOTICE

The text and drawings of this patent application are copyright © 2023 by Andrew Somers. All Rights Reserved except as specified below: A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

### Technical Field

This disclosure generally relates to psychophysics, and particularly that of the human vision system (HVS), and to perceptual characteristics such as color & contrast, and lightness, darkness, and brightness. These psychophysical attributes are applied to typography and graphic design in a practical way, to provide guidance to designers, allow automation of color selection, improve visual readability, and improve visual discernibility, particularly as it applies to dynamic documents on a self-illuminated displays or devices.

### BRIEF SUMMARY OF THE INVENTION

Visual contrast is an aspect of perception. Like other human perceptions it is context sensitive, and therefore eludes absolute measurement. Contrast can however be predicted within a reasonable range based on rational assumptions and expectations of various environmental and psychophysical factors.

A principal objective of the invention is providing the method and means to quantify human perception of contrast, particularly that of text and text-like design elements, and then use the subsequently derived perception data to instruct the implementation of methods and means to predict the perceived contrast for a given stimuli in combination with a given set of colors.

Further, this invention aims to do so in a perceptually uniform manner, such that a given numerical change in value is relative to the perceived change across the visual range of available colors on a self-illuminated display or device. Perceptual uniformity is very important in order to facilitate the development of automated color systems which are capable of adjusting colors without human intervention.

The ultimate goal is to provide substantially improved guidance to designers and content creators, and to improve readability and visual accessibility of text content displayed on self-illuminated monitors and devices for all visual users.

### BRIEF DESCRIPTION OF THE SEVERAL VIEWS OF THE DRAWINGS

FIG. 1: Protan Compensation Flowchart, showing the pre-processing stages prior to the lightness contrast calculation.

FIG. 2: An example formula of an embodiment of the present invention, this example is a reduction to practice of the principles of the present invention, referred to as the Accessible Perceptual Contrast Algorithm (APCA). FIG. 3A: This chart shows the processing of the input luminance, designated Yₛ, and the resultant lightness curves, using exponents which are dependent on factors such as the polarity. In this chart, the darker input luminance is set to 50% of the lighter input luminance, done in order to illustrate how the curves differ based on the context of polarity.

FIG. 3B: This chart compares various lightness curves as used in the present invention, against luminance (the straight line), and some other lightness curves, such as CIE L*.

FIG. 4: This is an example of the user interface for the contrast matching experiment software. The exponent controls allow experimental derivation of the exponents for creation of a contrast model that matches the empirical data.

FIGS. 5A, 5B, 5C, 5D: Example contrast matching experiment screens, showing the stimuli and the slider controls for adjusting the dark, middle, and light colors, as well as the control slider for the overall screen second proximal background, which we see is varied in these four figures, light grey in FIG. 5A, medium gray for FIG. 5B, dark gray for FIG. 5C, and black for FIG. 5D.

FIGS. 5E, 5F, 5G, 5H: Example contrast matching experiment configurations. 5E & 5F are examples of mixed polarity, 5G is an example of positive polarity only and 5H is an example of negative polarity only.

FIG. 6A: Contrast matching example, showing a stimuli pair with the middle color at different levels of adjustment.

FIG. 6B: A chart of the lightness of the middle contrast color from the contrast matching experiments relative to the setting for the overall screen second proximal background. This chart shows the effect of adaptation state and various polarities relative to the position of center contrast between black and white.

FIG. 7: The human contrast sensitivity curve relative to spatial frequency, with sample fonts of different sizes, and callouts indicating their approximate location on the sensitivity curve. All of the sample text fonts are at the same CSS color, demonstrating the substantial shift in contrast sensitivity as spatial frequency increases.

FIG. 8: Chart comparing results between WCAG 2 contrast criterion and the present invention, referred to on this chart as APCA.

FIGS. 9a, 9b: Spatial frequency examples, demonstrating that as spatial frequency decreases due to increasing stroke thickness, a substantially greater increase in contrast is observed.

FIGS. 10a,10b,10c: Screenshots of the user interface of a contrast calculator embodiment of the present invention, showing the color input areas and the dynamic example text display. The size of the text in the display changes in accordance with changing Lc contrast values.

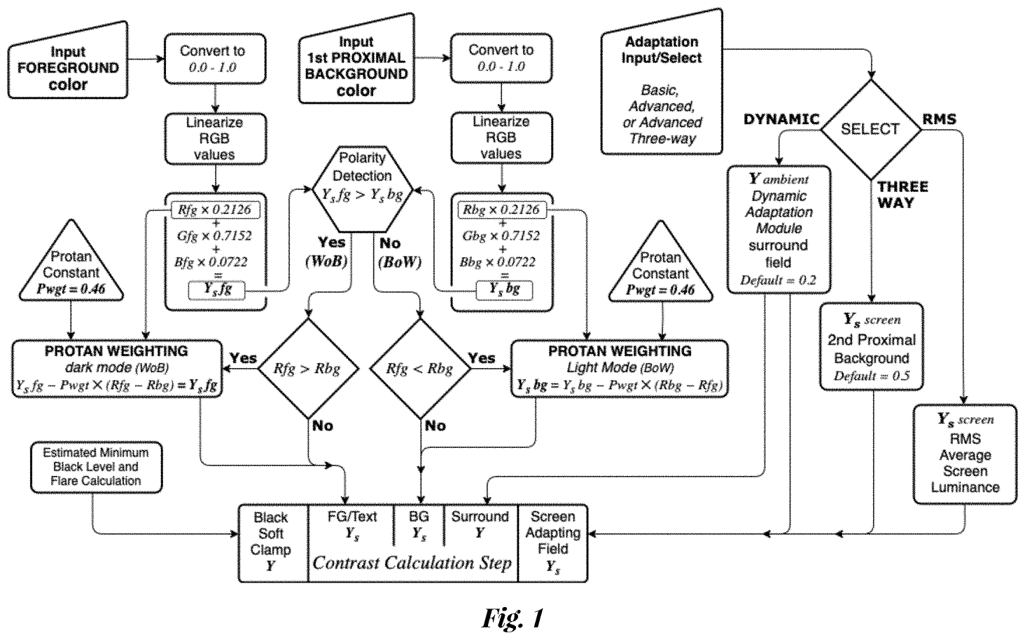

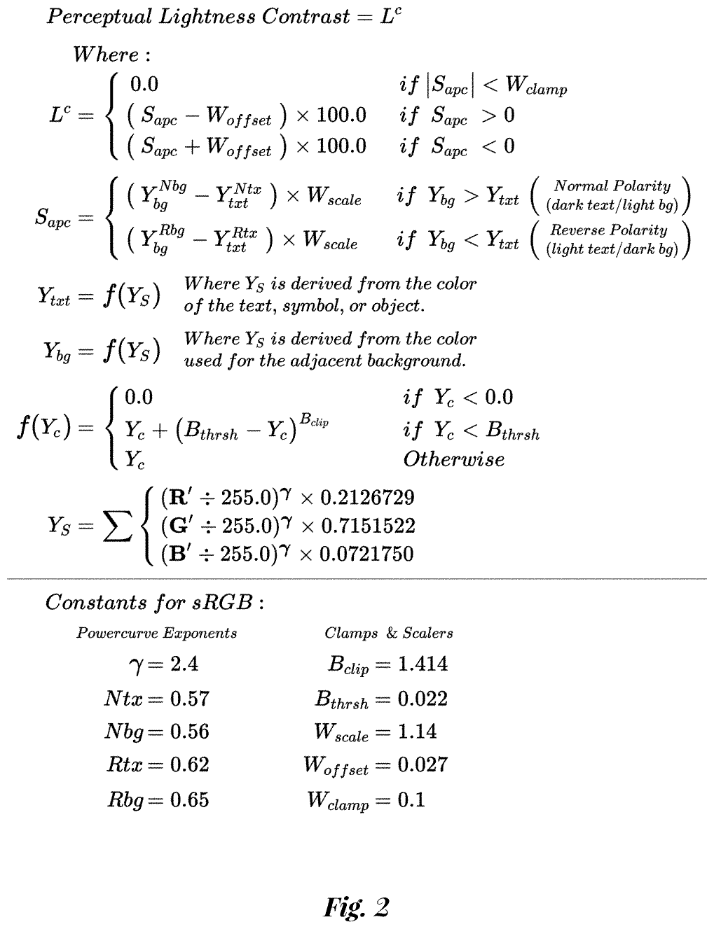

### TERMINOLOGY

This specification uses terminology that may be clear to those with a thorough understanding of the fields of color and visual perception. Nevertheless, the present invention spawned new terminology, so to ensure clarity, this brief glossary defines several key terms used herein.

### Human Vision System (HVS) Measurable Characteristics

Visual Acuity (VA)—acuity generally refers to the ability of the eye's optics to focus light onto the photoreceptors of the back of the eye (the retina). Acuity may also be impacted by ocular, neurological or other impairments. For a clinical evaluation, acuity is usually measured with an eye chart consisting of high contrast stimuli such as letters at varying sizes. Poor acuity is usually understood as blurry vision or an inability to focus, and the need for larger text or magnifying devices.

Visual Contrast Sensitivity (CS)—contrast sensitivity refers to the ability of the human vision system to discern the just noticeable differences (JND) between the visible and invisible. For a clinical evaluation, CS is measured by using a chart of large bold letters of uniform size and larger than the minimum acuity size, but where each set of letters are succeedingly lighter, therefore closer to the color of the background. CS is usually assessed relative to achromatic luminance. Color (hue sensitivity) is usually assessed independently.

Visual Color Discrimination (CV)—the ability to distinguish different wavelengths of light, or combinations thereof, as unique, individual hues. For a clinical evaluation, a color ordering task can be performed, where a test subject places a series of tiles, each with a unique hue, in the appropriate order for the given set of hues.

Visual Readability & Comprehension Assessment (VRCA)—a measure of the critical thresholds for best fluent readability and comprehension. For a clinical evaluation, Full paragraphs are read and an eye tracker is used to measure the saccades to calculate reading speed, and a follow-up test is administered to determine comprehension.

Critical Contrast—the supra-threshold luminance contrast, where increasing the contrast further will no longer result in an increase in reading speed or comprehension.

Critical Font Size—the text size measured as a visual angle, as rendered and read at a given distance, where increasing the size further no longer results in an increase in reading speed or comprehension.

Readability Contrast—a blanket term that covers the size, weight, spacing, and luminance contrast values as perceived in combination for visual reading.

### HVS in a Nutshell

As clinical measures, visual acuity and contrast sensitivity provide a good indication of overall visual function, especially for readability.

Visual acuity is measured as the minimum size of a letter or stimuli, usually presented near a maximum luminance contrast, that can be resolved and recognized.

Contrast sensitivity is measured as the minimum luminance difference of a letter or stimuli versus the surrounding background, with the letter presented larger than the visual acuity size, that can be correctly recognized.

Color Vision Deficiency (CVD, sometimes called color blindness) refers to problems distinguishing between certain hues. By itself, CVD does not impair overall visual function. Compared to normal or standard vision, those with CVD have equivalent or better acuity and contrast sensitivity. CVD usually has little to no effect on readability. One exception relates to protanopia, as the protan forms of CVD are insensitive to red, and therefore red against black has greatly diminished contrast. While CVD does not affect readability per se, color-related vision impairments can severely impact other visual tasks such as object recognition, cognitive understanding of color-coded information, and discerning between certain color combinations.

### Measurable/Calculable Quantities (not Perceptions of HVS)

These refer to physical properties that can either be measured, or calculated directly from physical measures, prior to perception by the HVS.

Luminance (Y or L)—achromatic visible luminous intensity, that is, the wavelength-weighted sum of the visible light without regard for color. L represents a physical measure of luminance, typically in units of cd/m². Y represents a relative measure of luminance, nominally between 0.0 and 1.0. A luminance value is mathematically linear (additive) to changes in luminous intensity, just as light is in the real world. Luminance is a physical quantity, but it is not the human perception of lightness. However, luminance as a quantity is spectrally shaped to account for different sensitivities of the HVS to different wavelengths of light.

Luminance Contrast—as used in this disclosure, this refers to the physically measurable difference between two luminance values. It is important to understand that luminance difference in and of itself does not define the human perception of contrast. Also, HVS contrast perception is largely tied to spatial frequency, more so than the difference between two colors or luminances. To be discussed below.

Color—as used in this disclosure, “color” refers to a unique hue or colorfulness (saturation or chroma), but disregarding lightness or darkness. While a specific wavelength or spectral distribution is measurable, color as a sensation is not a measurable absolute. Color perception can be modeled, see perceptual qualities below.

Spatial Frequency (SF)—Abstractly, spatial frequency is typically defined as cycles per degree (cpd) of the visual angle (vθ) of a stimulus, as subtended onto the retina of the eye. In a practical sense, SF refers to the measurable weight and size of a font, letter & line spacing, the stroke width of a line, or the size, shape, & padding of an interactive button or control. A thinner font or narrower stroke width is a higher spatial frequency than a bolder or thicker stroke, which is a lower spatial frequency. Higher spatial frequencies above ˜8 cpd require more luminance contrast to be visible than lower spatial frequencies (see “perceptual qualities”).

Visual Angle (vθ)—refers to the size of a given stimuli as subtended onto the retina of the eye via the nodal point of the eye. It can be calculated for a given distance to a given size object based on the following formula:

vθ=2*arctan(size/(2*distance))

Gamma or Tone Response Curve (γ or TRC)—a mathematical function that is commonly applied to signals, colors, or image data. Purposes include reducing perceived noise, improving data utilization, correcting for display nonlinearities, and adjusting perception relative to the adaptation state.

Electro-Optical Transfer Function (EOTF)—a function specific to a physical display's physical light output relative to the input color values. See also gamma.

Positive Polarity—dark text on a lighter background, also known as light mode.

Negative Polarity—light text on a darker background, also known as dark mode.

Estimated Screen Luminance (ESL)—the estimated luminous intensity at the face of a display for a given input color, derived by linearizing each color primary value by applying the gamma exponent and subsequently multiplying each result by its associated coefficient, then summing the products, where: The independent input values for each of the primary colors of the display are scaled to 0.0-1.0. The display's assumed gamma is as commonly adjusted for the assumed reference environment (e.g., 2.4), and the standardized color primary coefficients are those for the given display's color space.

Sampling Visual Fields for Test Inputs—not all of the following list of inputs are needed, as many can be estimated, assumed, or eliminated depending on the expected environmental, surround, or design conditions.

Stimuli Luminance—the text or non-text element, also referred to as the foreground luminance.

Stimulus Spatial Frequency—the font weight and size, or the graphic element border or outline thickness, and contributing spatial frequency characteristics such as white space.

First Proximal Field luminance—the immediate, adjacent background color around the stimuli.

Proximal Border Luminance and SF—when the stimulus has a border between it and the first proximal field.

Second Proximal Background Field Luminance—the larger background around the proximal field, and can be an important calculation for a graphic feature such as a button or the text stimulus is on the button, therefore the button is the first proximal field, but the button is then surrounded by the second larger proximal background. This can be a solid color background, or it can be the RMS (root mean square) averaged luminance of the total content on the display.

Surround Field Luminance—refers to the visual area around the display or device, including any bezel or case around the display or device itself. This information can be provided by an ambient light sensor.

Adapting Field—this is the ambient luminance encompassing the global illumination condition of the immediate environment, including the environment not immediately in the field of view. The ambient luminance determines the global light or dark adaptation. This can be provided by an ambient light sensor. The ambient luminance can also result in flare or glare on the surface of the display's screen, which is a separate but additive contribution to the screen's luminance.

Tacit Adaptation Value: For practical implementation purposes, we may wish to consider that an RMS average of the entire screen serves as a proxy, combining the background field, the surround field, and the adapting field.

### Perceptual Qualities Processed by the HVS:

Perceptions are not directly measurable in the absolute sense, as they are context sensitive sensations of the human vision system. Nevertheless, as neurological functions they can be predicted or modeled if the context and surrounding conditions of the stimuli are understood.

Color—as used in this document, color does not refer to the grey, achromatic lightness/darkness/brightness—as used herein, color refers to hue or uniqueness, and colorfulness or color purity.

Colorfulness—is the color intensity or purity, with common correlates known as saturation and chroma.

Hue—refers to a particular category of color sensation such as red, green, yellow, blue. Color, as in hue but disregarding lightness/darkness, holds very little detail, and in the HVS is processed at 33% of the resolution of luminance. Some people with color insensitive vision are unable to distinguish some hues. Coding information with different hues should never be the sole means of presenting that information.

Lightness (L*) (aka Lstar)—more formally, “Perceived Lightness”, is the perception of physical light intensity. In other words, where luminance is a mathematically linear measure of physical light, lightness is the estimated perception of that luminance. Some color models attempt to provide a mathematically uniform estimate of perception. One such symbol is L* which refers to CIE L*a*b*, and should not be confused with luminance L.

Brightness (Q) and Darkness—these are relative perceptions associated with lightness.

### Quantities and Qualities of Visual Contrast

Visual contrast is best thought of as a perception, and not as an absolute measure. While we can certainly measure the absolute or mathematical difference between independent colors, that by itself will not define the perceived difference between those colors.

Therefore, we can say that there is a “physical contrast” which means an absolute or physically measurable distance or difference, and a separate “perceived contrast” which is sensitive to context and viewing conditions, and is a perception that is not directly measurable, though it can be modeled and predicted.

There are many forms of contrast, and the different types of contrast interact with and are affected by each other as well as affecting and being affected by other aspects of vision, not to mention design characteristics. Visual contrasts divide into four broad categories:

Luminous contrasts (light)—Measurable: luminance, perceptual: lightness/darkness/brightness.

Chromatic contrasts (color)—Measurable: wavelength and spectral distribution, perceptual: hue, colorfulness (chroma, saturation).

Spatial contrasts (size/distance).

Temporal contrasts (time/motion).

### Luminous Contrasts (Light)

Luminance contrast: measured difference between two or more luminance values. This can be described with simple linear additive math, or as a simple ratio.

Lightness contrast (Lc): the perceived lightness/darkness/brightness difference between two or more elements (such as text and the adjacent background). Lc is a particularly important form of contrast for the fluent readability of text, as the HVS perceives small details almost exclusively through achromatic luminance, and not color (hue/colorfulness), which is a third the resolution of luminance. The vast majority of fine details are held in the luminance channel, i.e., the lightness/darkness part of an image or design, disregarding color as in hue. While perceived lightness contrast is in part a function of luminance differences, it is spatial frequency characteristics, particularly line or stroke weight or thickness, that are more likely to have a pronounced effect on perceived lightness and readability contrast.

### Chromatic Contrasts (Color)

Spectral contrasts: the measured difference between two or more spectral distributions of visible light. The metameric aspects of HVS can allow two different spectral distributions to appear to be the same perceived color. As with luminance, this physically measured contrast does not define the actual perception.

Color contrasts: the perception of the difference between two or more spectral distributions of light. Hue/color contrasts are three times weaker than luminance or lightness contrasts. Color, as in hue and disregarding lightness/darkness, holds very little detail. Some people are insensitive to some hues, therefore contrasts of hue should never be the sole means of presenting information. However, hue can be very effective as an added contrast for data-viz, or as an information organization element.

Spatial contrasts: contrasts of font weight, size, thickness, position, spacing/whitespace. Spatial contrasts have a direct effect on the perception of lightness and color contrasts. In many cases, a seemingly small spatial change can result in a much larger change in perceived lightness contrast, as opposed to a change in luminance. For instance, the difference between a bold or normal weight reference font is essentially equivalent to a lightness contrast change of Lc±30.

Temporal contrasts: contrasts of time, speed, direction, and state change are not a significant part of the present invention.

### Discussion of the Art:

### A Brief History of Readability Contrast

For much of the last thousand years of the printing press, texts were printed using dark or black ink on a white or very light medium, such as parchment, linen, or paper. As far as luminance is concerned, black text on white is the maximum practical print contrast, and black ink remains the ubiquitous standard for printing text on an offset press today.

But this is not to say that the perceived contrast cannot be adjusted prior to actual printing. If a graphic artist needs to modulate the contrast of printed text, they can do so by choosing a lighter weight (thinner) or heavier weight (thicker) font from the given font family.

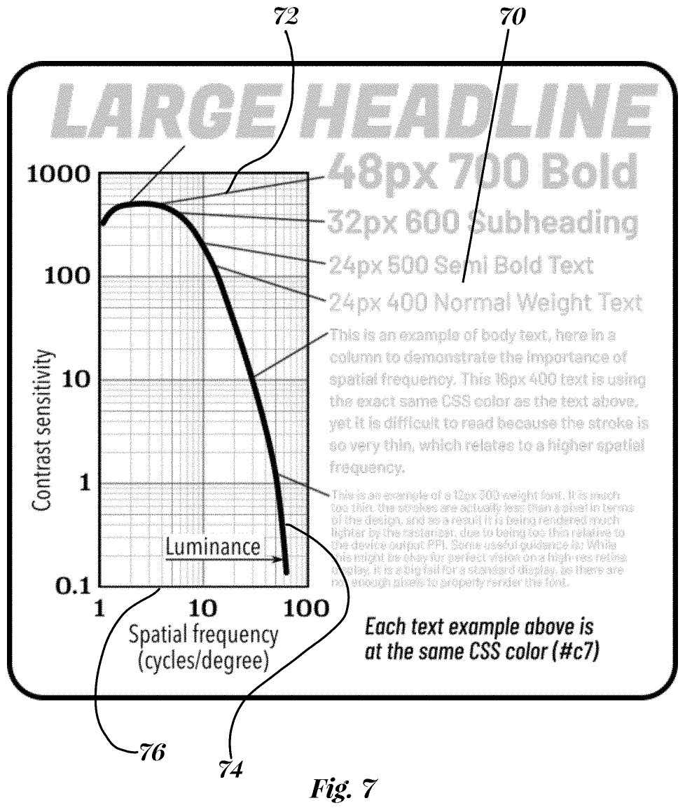

Human perception of visual contrast is largely a function of the spatial frequency of the stimuli, where higher spatial frequencies, above ˜8 to 10 cycles per degree of visual angle, are associated with rapidly decreasing contrast sensitivity. Therefore, a thinner font can be expected to appear lower in visual contrast than a thicker, heavier weight font. This is demonstrated in FIG. 7, using example fonts 70 all of the same color grey, but at different sizes and weights. Each sample font has a straight lead line 72 that touches the contrast sensitivity curve 74 at the approximate spatial frequency 76 of that particular sample font.

Font characteristics notwithstanding, in the art of physical printing, the measurable luminance reflectance value difference between the black ink and white paper remains essentially unchanged and locked in place, not adjustable as it is with the dynamic content on a computer display.

Throughout the centuries of physical printing, trial and error experimentation evolved into classical design guidelines. Luminance contrast was virtually static and near maximum, so there was no compelling need for a perceptually uniform contrast formula to provide guidance to designers. A skilled designer could make judgements on readability that would be fixed in place—and in contrast—after printing.

Because the skill of the pre-press design staff could ascertain color or contrast problems before printing, and the printed version would be static, an automated mathematical model of contrast perception was not a tool a graphic artist needed for any practical reason. That is, until recently.

### The Unreadable Revolution

According to Pew Research, the year 2004 marks the start of the very rapid decline in traditional-print newspaper revenue (Pew Research Center Newspapers Fact Sheet June 2018).

Circa 2004 was also a time of rapid growth of the Internet. WiFi and Internet connectivity was not only getting faster, it was the year the first WiFi enabled devices like PDAs and 3G web-capable cell phones became available. And 2004 saw the birth of social-media giant “Facebook”.

Over the succeeding years, newsstands and book stores vanished, as digital media, distributed over the internet, replaced traditional print media. The consequence to society is that readership has decreased, and visual fatigue from reading on displays and devices has increased, a phenomenon referred to as Computer Vision Syndrome or Digital Eye Strain.

Circa 2008, the World Wide Web Consortium, an organization setting standards for web-based content, published the WCAG 2.0 accessibility guidelines. Among the listed guidelines, WCAG 2.0 specifies a luminance contrast for text. Unfortunately, the math and methods used to estimate contrast per WCAG 2.0 are not well suited to modern, advancing technologies such as high-definition displays, and automated color tools. A key reason here is that the calculated results from WCAG 2.0 contrast lack perceptual uniformity, and the associated guidelines are missing essential spatial-frequency considerations.

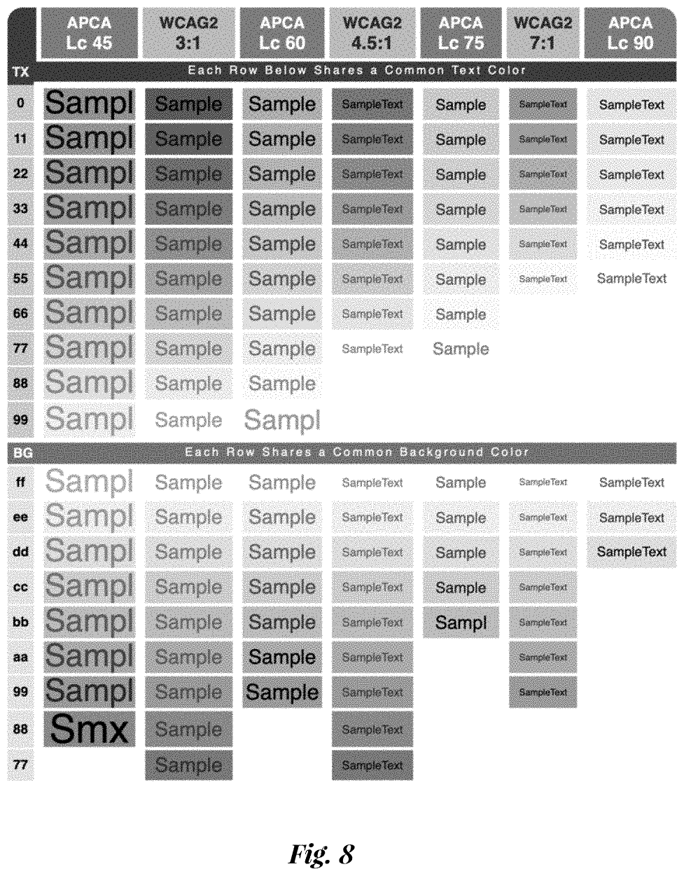

Relative to a perceptually uniform model, some studies have found that WCAG 2's uniformity error can be as high as ±250%. The inaccurate results can be particularly harmful to readability for some common forms of color vision impairment (aka colorblindness), as some useful colors are incorrectly rejected, while many related colors that are inaccessible are incorrectly allowed. See FIG. 8 for a chart of WCAG 2 contrast results, compared to an embodiment of the present invention referred to as APCA.

One unintended consequence is confusion for what some have called arbitrary or conflicting design criteria, and poor end results. In conjunction with the contrast thresholds chosen, the guidelines allow websites to use unfortunately low contrasts for primary content text. A recent survey of archival site records of major websites found they once used good, high contrast text, but in recent years have softened it to a low-contrast gray that is difficult to read and can increase eyestrain.

Another serious problem is the absence of adequate spatial considerations. Spatial frequency (SF) in a design context refers to font weight, size, line thickness, and so on. SF is the primary driver of visual contrast perception. with the human vision system perceives smaller and thinner stimuli with a significantly lower contrast sensitivity. Compared to a 24 px bold font used as a headline, a column of 12 px regular text stimulates 200 times lower contrast.

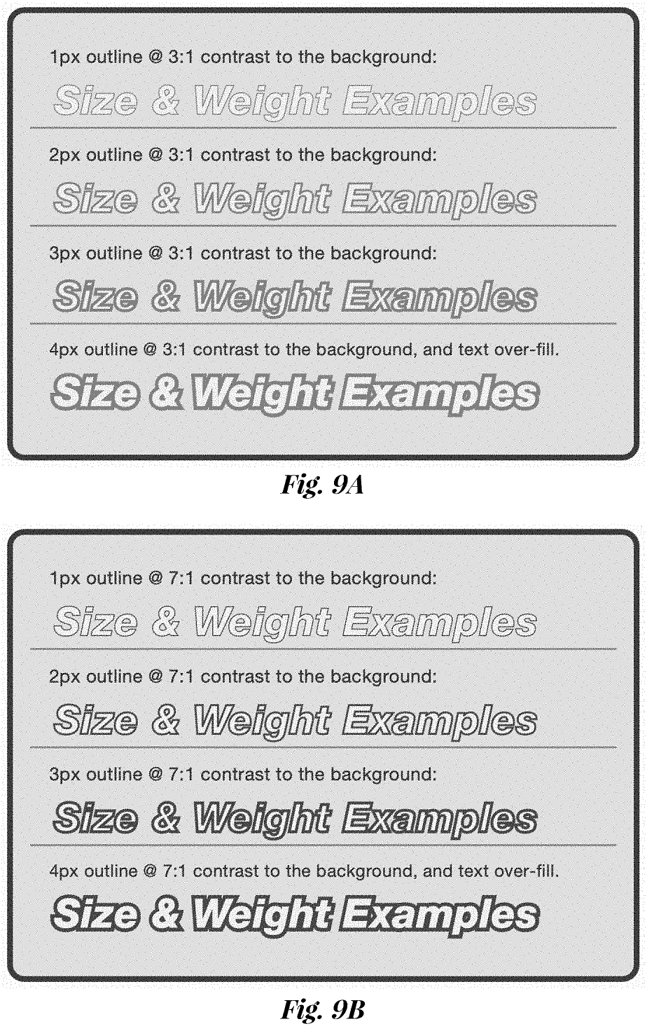

Examine FIGS. 7, 9A, and 9B for demonstrated examples of the importance of spatial characteristics when discussing contrast and contrast design guidelines.

### Automatic Failure

The non-uniformity of WCAG 2 contrast makes it unsuitable for use with automatic or autonomous color manipulation, color palette generation, or color shifting operations. WCAG 2's contrast flaws require human intervention to ensure that selected colors are appropriately useful.

For automatic or autonomous use, a color or contrast model must be perceptually uniform, so that automated color manipulations using the model have predictable results without human intervention. The present invention was developed to address these specific problems, as well as to promote better readability and visual accessibility for all.

### The Preferred Embodiment of the Present Invention

The present invention produces a contrast value that is perceptually uniform over the display's available color and contrast range. For instance, changing the lightness contrast by Lc 15 appears essentially the same, regardless of where in the available range that change is applied.

This allows thresholds for guidelines to be meaningfully useful across the available visual range, without the need to use brute-forced guidelines which are only valid for a small part of the available contrast range,

The perceptual uniformity of the present lightness contrast invention uses simple math for efficient and easy integration into existing design systems and contrast calculators. The present invention: Can be adjustable to fit specific environments or use cases.
Can accommodate different color spaces.
Can therefore be integrated with autonomous color manipulation systems.
Can be used to invert colors to automatically change light mode into dark mode, or other modes or color schemes.
Is extensible for adding processing modules or features. For instance, the Color Vision module adjusts the contrast value result to ensure that protanopia (red insensitive vision) does not lose readability. Additional color inputs can be provided for the surrounding background screen color, total screen color, and ambient illumination, allowing calculation of a dynamic, estimated adaptation, and subsequently more accurate contrast prediction.
Is polarity aware of light mode vs dark mode to further improve accuracy.
Is spatial frequency aware, which both expands design flexibility and improves readability. Lc thresholds can be lower for low spatial frequency elements, allowing more color choices. Lc thresholds are higher for HSF, such as body text, improving readability.

Use-case and font suggestion module is separated so that it is easy to modify for different languages, writing systems, and/or applications of use.

### Summary of Key Contrast Science

### Weber Contrast:

In 1860, Gustav Fechner published “Elements of Psychophysics”, containing Weber contrast, a simple formula based on the Weber/Fechner law, that attempts to define and predict the perception of visual contrast.

WeberContrast=(Yhi−Ylow)/Yhi

The contrast value is defined as the difference between a high luminance and a lower luminance, divided by the higher luminance.

Weber contrast is at best a rough approximation. And importantly, Weber contrast is not perceptually uniform across the visual range. This means that a given change in value does not equate to the same actual change in perception across the visible range. Weber contrast is mainly useful at the finding the threshold, or “just noticeable difference” (JND) between the stimuli and its surround.

### Stevens Et Alia

In 1957, Stanley Stevens published a body of psychophysical data, supporting theories relating to the use of a power law to define the relationship between stimulus intensity and perception. Among his findings included a shift in exponents for the power curves based on changes in the spatial frequency of the stimuli.

“Apparent contrast as a function of modulation depth and spatial frequency: A comparison between perceptual and electrophysiological measures” (Franzen and Berkley 1975, journal: Vision Research) finding that the power curves that defined contrast perception used higher exponents with increasing spatial frequency stimuli.

In 1981 the journal Vision Research published “A Power Law for Perceived Contrast in Human Vision” (J. Gottesman, G. Rubin, and G. Legge, Vision Research Vol. 21. pp. 791 lo 799. 1981). This is one of the several papers that demonstrated the usefulness of the power law in terms of defining perceptual contrast curves.

Much of the experimental work was conducted using sinewave gratings in several of these papers. Michaelson contrast is often used to do the contrast calculation, which is:

MichaelsonContrast=(Yhi−Ylow)/(Yhi+Ylow)

This is a useful equation in research for assessing contrast of different spatial frequencies, however it is not perceptually uniform over the visual range, and therefore lacks usefulness for design guidelines for text on a background.

### Munsell, CIE, and Delta L*

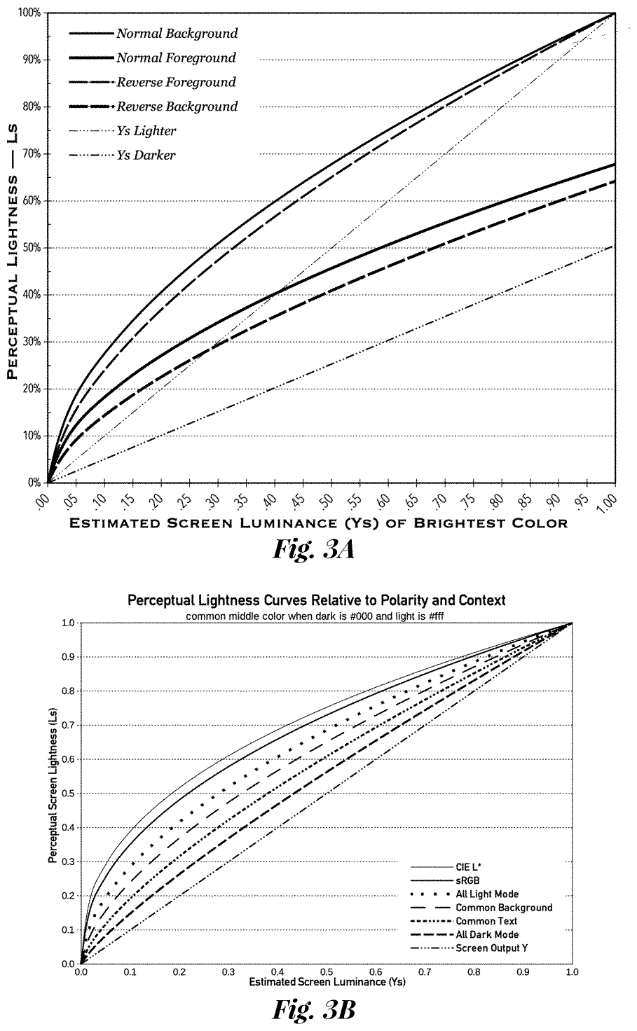

The Munsell color ordering system does not define a contrast equation per se, but it does provide for empirically derived perceptual lightness values. In 1976 the CIE standardized the CIE L*a*b* and CIE L*u*v* color spaces, both of which feature L* aka Lstar, a quasi-perceptually-uniform lightness value that is based on Munsell value. See FIG. 3B.

L* is a power-law based perceptual lightness curve, similar to those proposed by Stephens. Luminance, Y from CIEXYZ, is converted to L* perceptual lightness with the following equation:

if Y>0.008856 then: L*=Y\hat{ }0.333*116−16 else L*=Y*903.3

This method provides a version of perceived lightness, but not perceived contrast. Nevertheless, some have converted a pair of colors to their respective L* values, and then found the difference, and used the difference result as a contrast metric.

Extensive evaluation by this author in 2019 showed that the L* difference, which we will refer to as delta Lstar (ΔL*), calculated a contrast value that is not functionally different from WCAG 2.0. As a result, ΔL* suffers some of the same failings.

### Barten's Spatio-Temporal Model

In 1993, P. Barten presented his “Spatio-temporal model for the Contrast Sensitivity of the human eye and its temporal aspects”. Proc. SPIE 1913-01, and further in 1999 Barten's book “Contrast sensitivity of the human eye and its effects on image quality” Barten, P. G. J. (1999).

Barten's model considers neural noise, lateral inhibition, photon noise, external noise, limited integration capability, the optical modulation transfer function, orientation, and temporal filtering. It is one of the more complete models of contrast perception, however it is also very complex and not well suited to public policy guidelines.

### Color Appearance Models

In 1995 M. Fairchild introduced RLAB, a modification of CIELAB which provided for adjustable exponents based on surrounding illumination conditions. The idea of varying the exponents of nonlinear perception functions, in relation to dark, dim, or normal surround illumination is also seen in 1997 with Luo, Hunt in CIECAM97, and in 2002 with Moroney, Fairchild, Hunt, Li, Luo, Newman in CIECAM02.

These color appearance models provide for finding a given color difference between two colors. However, they generally do not take into consideration spatial characteristics, and do not necessarily reflect human perception of contrast of text, which is typically at a high spatial frequency.

### WCAG 2.0

Circa 2005-2008, the World Wide Web Consortium developed and published WCAG 2.0, the Web Content Accessibility Guidelines. WCAG 2.0 provides a simple formula for contrast, along with related guidelines specifying certain contrast ratio thresholds in conjunction with the use of text on a webpage or similar electronically displayed document.

WCAG 2 contrast is calculated in two stages. In stage one, the sRGB color values for a first input color and a second input color, are individually converted to their theoretical relative luminances Y1 and Y2, using the piecewise sRGB to CIE XYZ formula as defined in the sRGB standard, IEC 61966-2-1:1999.

Each luminance in a pair has a flare value of 0.05 added, and the higher luminance is then divided by the lower luminance, to create the WCAG 2.0 contrast ratio.

WCAG2Contrast=(Yhi+0.05)/(Ylow+0.05)

While WCAG 2 contrast is a minor improvement over the classical Weber contrast, it is still not perceptually uniform for predicting the contrast of text or other high-spatial-frequency elements presented on self-illuminated monitors, especially in typically bright office environments.

In particular, WCAG 2 grossly overstates the contrast value for very dark colored pairs, in some cases by ˜250%. This results in potential false passes by the WCAG_2 math, and further shows that WCAG_2 cannot be used to correctly calculate contrast for colors needed for “dark mode” color schemes, which is where the text is lighter than the background.

### Modified Weber

(U.S. Pat. No. 10,755,673 B2)

In 2016, A. Hwang & E. Peli proposed the “Positive and negative polarity contrast sensitivity measuring app” (IS&T Int Symp Electron Imaging).

The Hwang/Peli Modified Weber provides a slightly better assessment of contrast as it applies to computer monitors/sRGB than the WCAG 2 method, and it is similar:

HPmodWeberContrast=(Yhi−Ylow)/(Yhi+0.05)

The present author also conducted evaluation and empirical testing of this method in 2019, and while it was a modest improvement relative to WCAG 2.x, it's still lacking the level of perceptual uniformity that is needed for certain applications, such as automated color and design systems.

This author developed a further modified Weber derivation, adjusting the flare value recited by A. Hwang & E. Peli, and scaling:

AMSmodWeberContrast=((Yhi−Ylow)/(Yhi+0.125))*0.8

Despite several different offsets in scaling's, these modified Weber contrasts did not provide substantial matching to the empirical suprathreshold contrast perception data in studies conducted by this author.

### Phi-Tuned: DeltaPhiStar (DPS)

In connection with the early investigations from 2019 to 2022, this author discovered that a modification to the above mentioned ΔL*, using the constant phi, would significantly improve the useful results in the middle range, which is most useful for design guidelines. Assuming a background and a text color are converted to standard D65 L* as bgLstar and txLstar, then:

dpsContrast=|bgLstar\hat{ }1.618−txLstar\hat{ }1.618|\hat{ }0.618*1.414−40

Here, after converting the colors to L*, raise each using phi as the exponent, and find the absolute value of the difference. Then apply the inverse exponent 1/phi (0.618) to the result, and finally scale it by multiplying by 1.414, and subtracting 40. These exponents and scaling produce a reasonably uniform result between Lc 45 and Lc 75, which is the key range for text. The main drawback with DPS contrast is that for contrasts lower than Lc 45 or higher than Lc 75, there is a decline in perceptual uniformity.

### RELATED DISCLOSURES OF THIS INVENTION

Why APCA The Accessible Perceptual Contrast Algorithm: an embodiment of the present invention in practical use. “Why APCA” provides additional background and practical application to design guidelines. A. Somers, Mar. 20, 2022. URL: gitapcacontrast.com/documentation/WhyAPCA

“APCA Web Tool” is a technology demonstrator and reduction to practice of the predictive aspects of the present invention. A. Somers Mar. 20, 2022. URL: apcacontrast.com

### Contrast and Color for Design

An article by the inventor provides an encapsulated, comprehensive overview of color and contrast, including practical uses for the present invention.

Reference: “The Realities and Myths of Contrast And Color” A. Somers Sep. 6, 2022. URL.www.smashingmagazine.com/2022/09/realities-myths-contrast-color/

### ADDITIONAL REFERENCES

### Critical Size and Contrast

Several readability researchers, including S. Whittaker, J. Lovie-Kitchin, I. Bailey, G. Legge, have researched and defined acuity reserve, contrast reserve, critical size, and critical contrast. Selected References:
Whittaker S G, Lovie-Kitchin J. Visual requirements for reading. Optom Vis Sci. 1993 January; 70(1):54-65. doi: 10.1097/00006324-199301000-00010. PMID: 8430009.
Lovie-Kitchin, J., & Whittaker, S. (1998). Relative-size Magnification versus Relative-distance Magnification: Effect on the Reading Performance of Adults with Normal and Low Vision. Journal of Visual Impairment & Blindness, 92(7), 433-446. https://doi.org/10.1177/0145482X9809200704

### Color Vision and Contrast

Most individuals with color vision deficiency have otherwise normal or better visual function, and in particular good luminance contrast sensitivity.

### REFERENCE

“Contrast sensitivity of patients with congenital color vision deficiency.” C. Ilhan, M. Sekeroglu, S. Doguizi, et alia, Int Ophthalmol 39, 797-801 (2019). doi.org/10.1007/s10792-018-0881-7

### High SF

At high spatial frequencies, contrast sensitivity drops as luminance drops. For instance, roughly speaking, at an SF of 16 cpd, a 10× drop in luminance results in an approximate 0.5 log drop in CS. Reference:
“Measurements of achromatic and chromatic contrast sensitivity functions for an extended range of adaptation luminance”—K. Kima, R. Mantiukb and K. Leea a) Dept. of Radiation Applied Life Science, Seoul National Univ., Korea; b) School of Computer Science, Bangor Univ., United Kingdom

### JND and Readability

Threshold contrast sensitivity is not necessarily an effective predictor of supra-threshold contrast perception. Further, we find the contrast measures that define the threshold JND are not perceptually uniform when used at supra-threshold or at high spatial frequencies. Reference:
Andrew M. Haun and Eli Peli “Complexities of complex contrast”, Proc. SPIE 8292, Color Imaging XVII: Displaying, Processing, Hardcopy, and Applications, 82920E (24 Jan. 2012); https://doi.org/10.1117/12.915365

### U.S. Patents

The A. Hwang & E. Peli modified Weber is covered by patent no.: U.S. Pat. No. 10,755,673 B2 Date of Patent: Aug. 25, 2020. Nevertheless, our empirical studies found it to be insufficiently uniform per human perception. Also, the scope, intended application, and particularly the functional structure of the modified Weber, is distinctly different than that of the present invention.
U.S. Pat. Nos. 5,721,792, 7,912,289, 8,074,168, 8,144,979, 8,406,528, 8,411,987, 8,487,786, 8,520,022, 8,572,549, 8,699,815, 8,891,874, 8,929,679, 8,977,051, 10,264,266, 10,319,116, 10,643,353, 10,755,673, 11,062,435, 11,107,258, 8,891,874 B1—Legibility analysis of text in an electronic document.

### Note Regarding Prior Art

This brief overview of selected, notable prior art in the area of visual contrast was intended to cover the key science and established methods, but there are certainly many more. Nevertheless, empirical evaluations have shown that these existing models tested are not perceptually uniform for text & background presented on a self-illuminated display, which is a principal feature of the present invention.

### DETAILED DESCRIPTION OF THE INVENTION

### Objective

A principal objective of the invention is providing the method and means to quantify perceived contrast, particularly for medium to high spatial frequency stimuli at supra-threshold levels, such as text and text-like design elements, as seen by human test subjects. And further to use the subsequently derived study data to instruct the implementation of methods and means to predict the perceptual readability contrast for a given stimuli and set of colors, and to do so in a perceptually uniform manner across the visual range of available colors on a self-illuminated display or device.

### Quantifying Perception, an Overview

The present invention comprises a method and means to measure the perceived visual contrast of stimuli at various spatial frequencies, presented with different luminance and color combinations, and within the context of different design and environmental conditions. The method is particularly well suited for predicting text contrast on self-illuminated monitors and device displays.

Measuring supra-threshold contrast perception is accomplished by means of contrast matching two or more sets of visual stimuli, wherein a human study subject adjusts one or more colors to affect a visual match between the presented stimuli sets. The matching condition is met when the study subject achieves an equivalent visually perceived contrast and equivalent effective readability among the visual stimuli, as assessed by the study subject's own visual perception, in a defined environment

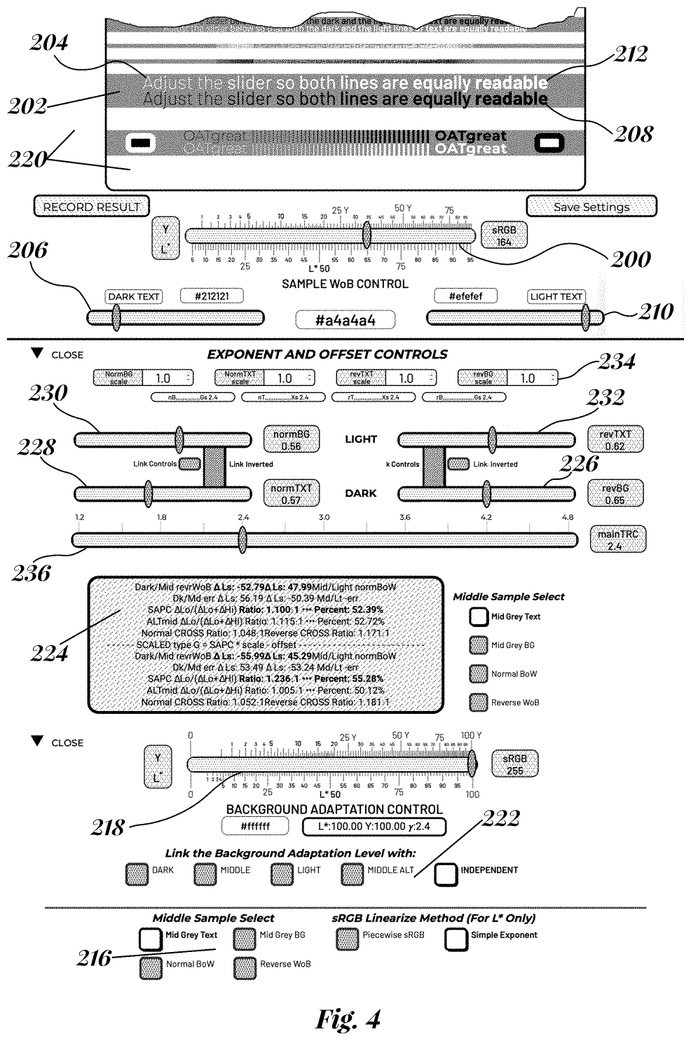

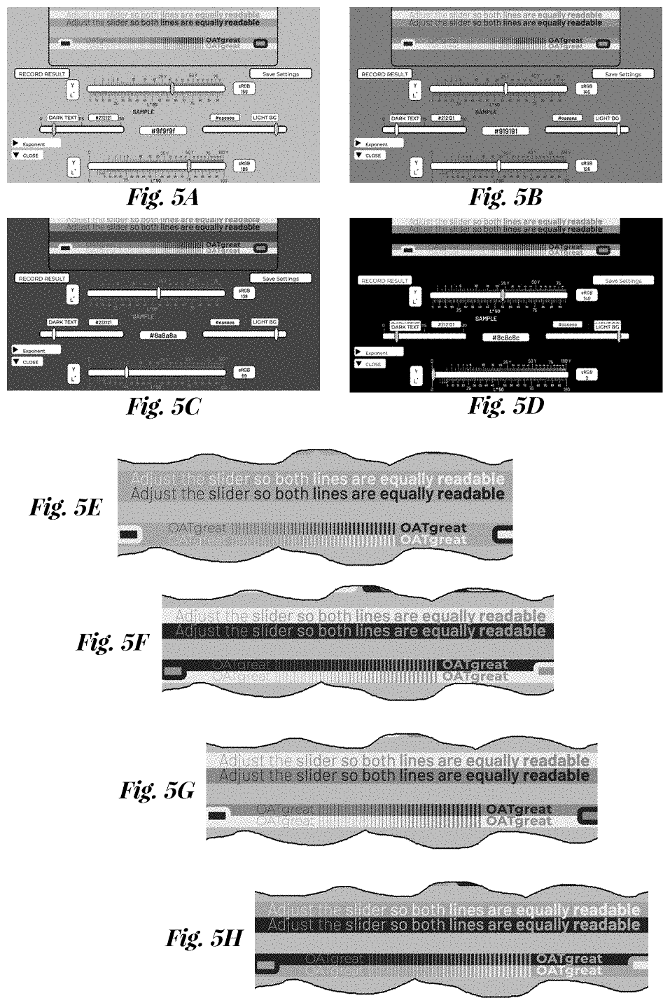

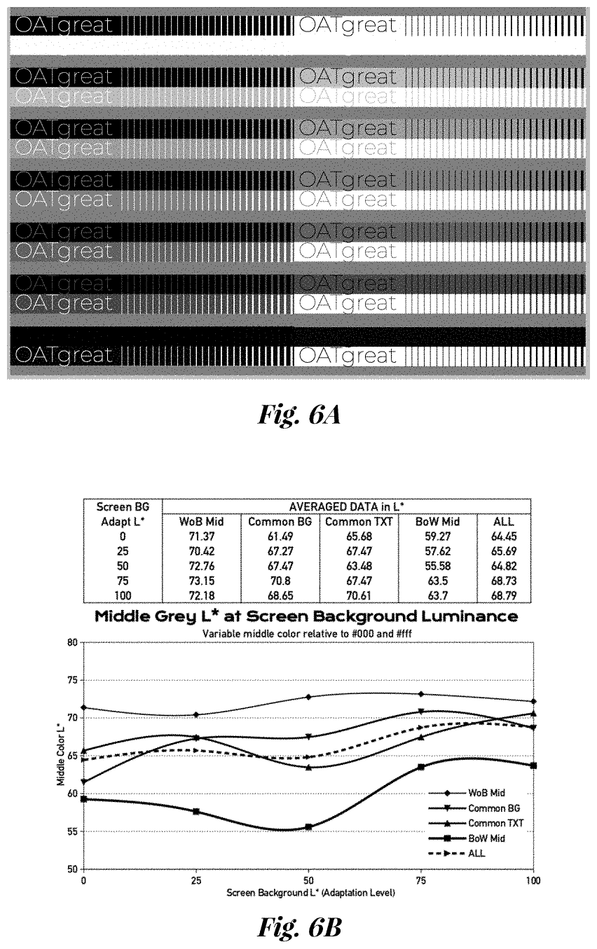

One preferred embodiment utilizes a plurality of lines of sample text in a range of sizes and weights, and non-text graphics comprising a range of spatial frequencies. See FIGS. 4, 5A, 5B, and 5C as well as FIG. 6A for examples of high spatial frequency stimuli configured as used for contrast matching experiments.

In the embodiment shown in FIG. 4, the top most variable slider 200 controls the common middle color 202 of the test sample texts 204 presented on the display, while the smaller slider on the left 206 controls the dark color 208 and the slider on the right 210 controls the light color 212. These small sliders also define the extent of available range of the middle slider. So, if the small dark slider is made lighter or the small light slider is made darker, the middle color will be limited to these extents. However, the middle slider itself always maintains its full range of motion, so as the color range becomes smaller the resolution of control on the middle slider becomes greater. In this embodiment the graticule 214 that surrounds the middle slider expands or contracts as needed to indicate the full range of color available for the middle slider.

Lower in FIG. 4, you'll see four buttons 216 under the label “middle sample select”. These buttons determine the mode test configuration regarding which lines of text are in light mode or in dark mode, and obviously this controls which color is linked to the middle control.

The numerical data of the stimuli parameters, colors, numerical color data, and/or luminance values, and the adjustments made by the test study subject, are recorded to a non-transitory computer-readable storage medium, and this data is subsequently used to define the perceptual curves that relate to that specific study subject's contrast perception characteristics, for later evaluation or aggregation with other study subjects.

The environmental and design conditions used during testing are chosen to replicate a reasonable selection of real-world conditions of when, where, and how content might be viewed. This is done to create perception curves that are most relevant for the expected use cases. Looking again at FIGS. 4, 5A, 5B, and 5C, Notice that the overall screen adapting background can vary from black to full white and anywhere in between. This is done during experimental trials to set the light adaptation state of the test subject. The large slider 218 at the bottom of the interface is the independent adaptation control which controls the second proximal background 220 that encompasses the majority of the screen, except the very small narrow areas that hold the stimuli, where the stimuli is surrounded by the first proximal background The independent adaptation control can be independently linked with any one of the other available test colors using buttons 222, such that it will follow that color if that color is adjusted.

The data sets from multiple test subjects can subsequently be aggregated and averaged to create a general purpose, perceptually uniform contrast perception curve which defines human perception of contrast relative to the context presented in the experimental conditions. Mathematical models are then adjusted to fit the aforementioned perception curve, and these models are further used to create algorithms, software tools, practical guidelines, and other useful means and methods, which can predict perceived contrast and thereby anticipate how readable and accessible a given stimuli and associated colors will perform in the context of a final design or presentation.

The resultant tools and guidelines can provide design recommendations to designers and developers regarding the use of specific colors, text sizes & weights, and other visual design choices.

### Creating and Using a Contrast Model

The method for developing a practical contrast model and guidelines is comprised of the steps including:

Measure the perception of contrast. Experimentally measure the perception of contrast using a plurality of human test subjects. Measurement experiments should be designed per the intended context where the model will be used, where the context is the colors or luminances input to create visual stimuli, and the surrounding illuminating conditions. Aggregate the test results together to create a standard observer of contrast within the contexts measured, wherein a given set of inputs is mapped to an expected perception.

Create a mathematical model by fitting output results to the standard observer, for a given set of color inputs. This model can be simplified by limiting its output results to the range needed for useful design guidelines of a specified scope. For instance, if the scope is contrast for fluently readable text, then the contrast model can be designed to support the specific range needed for text. This allows for the advantageous simplification of the math and therefore algorithm needed to reduce the model to practice.

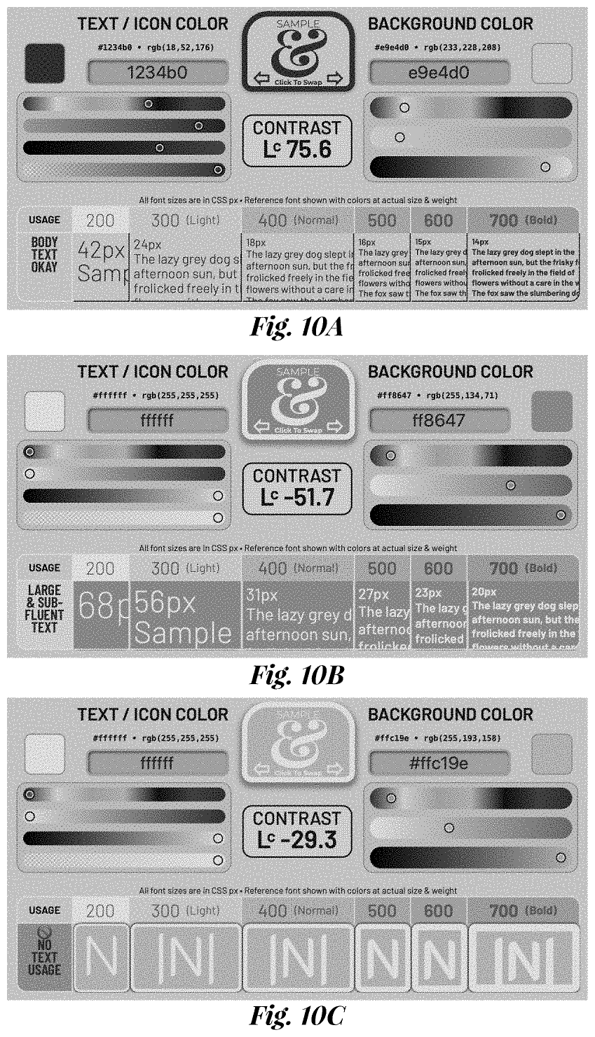

FIG. 3B is a plot of some of the characteristic perception curves of an embodiment of the present invention, FIGS. 10A,10B, and 10C, show and embodiment of one possible user interface of the present invention, which presents a plurality of sample stimuli to demonstrate how a given contrast selection not only will appear in practice, but providing useful suggestions in terms of minimum font size and weight.

Associate output results from the model with specific visual needs for a design. For instance, in terms of readability, specifics such as font size and font weight can be directly associated with a perceived contrast value, either with a lookup table or an algorithm, to permit a given level of readability. Determine the critical thresholds by referencing empirical research which experimentally defined the critical values needed for a given set of guidelines.

Create tools, documentation, and an effective set of guidelines for designers and developers. Such guidelines can go much farther than simply specifying a specific contrast, and as current embodiments are demonstrating, the tools can provide user selectable sample references that update in real time as colors and contrast change.

### Practical Perceptual Uniformity

Beyond design guidance, the perceptually uniform nature of the present invention is important for use with automated color selection, automated color palette systems, and dynamic user personalization features, such as creating automatic dark modes from a light mode, or contrast enhanced modes.

Historically, perceptual uniformity within a visual contrast model has been difficult to obtain, and an elusive goal of vision research. The present invention achieves uniformity through multiple stages of processing, taking a plurality of colors within a given color space that is related to a physical self-illuminated display or device.

In any preferred embodiment, each available or supported color space would have its unique math, method, and coefficients readily available, stored in such a way as accessible by the relevant computer code when needed to allow calculating the estimated screen luminance on demand.

The preferred embodiment includes means to individually select the desired color space from a plurality of available color spaces, therein employing the contrast calculation on the numerical values of colors relative to the given color space.

Preferentially, a color space related to the physical destination display is selected. Means are provided to convert color values using the selected color space to an estimated screen luminance value (ESL) by transforming the numerical values of the primaries for a given color to an estimated screen luminance.

If the color space is not so identified, a default color space is used. For web-based content the default color space would nominally be sRGB, as that is the standard for the World Wide Web. However other default color spaces could be used in a given system or application context.

For the preferred embodiment example, we will use sRGB, which employs a separate intensity value for each of the red, green, and blue channels. The ESL is calculated considering the destination display's EOTF characteristics (Electro-Optical Transfer Function). This is also known as the effective gamma or tone response curve (TRC), where the gamma is applied to each of the red, green, and blue values to linearize the values for further calculations.

Appropriate primary-color spectral weighting coefficients are provided for calculating a luminous efficiency function for the selected color space, and used as needed to calculate an ESL from the primaries.

The gamma or TRC used in the calculation, as well as the primary coefficients, may be adjusted to non-standard values to match with expected or measured display characteristics. Said adjustment may be pre-calculated, or adjusted dynamically as needed, such as for protanopia compensation, or in response to the current viewing conditions as determined by ambient light sensors.

The polarity of the luminance relationship between a pair of adjacent colors is important as it substantially affects the perception of contrast by the HVS.
A normal or positive polarity is when the adjacent proximal background color is lighter than the stimuli or text color.
A reverse or negative polarity is when the adjacent proximal background color is darker than the stimuli or text color.

For practical purposes, we consider the text to be the stimuli of interest. For non-text elements, we consider the color of the smallest element of a pair of elements to be the stimuli color. For example, a line or a border around an element would be the non-text stimuli. If that line or border is adjacent on one side to a first proximal color and on the other side to a second proximal color, then that line or border has two different contrasts, one for each separate color. Further, the perceived contrast between the first proximal color and the second proximal color is influenced by the presence and spatial characteristics of the line between them.

If a color adjacent to a stimulus is a gradient, then we would say that the contrast is a gradient contrast, which encompasses a range of contrast values.

### The Contrast Algorithm

The preferred embodiment of the contrast algorithm will accept a text color value and a proximal background color value, convert them to an estimated screen luminance (ESL), pre-process the luminances such as clamping low levels near black, and determining the polarity, then apply the text exponent for the determined polarity to the text luminance and apply the background exponent for that polarity to the background luminance, then subtract the text value from the background value, and finally apply the scale factor and/or offset, to create the final lightness contrast value.

The final lightness contrast value can then be used to indicate a minimum spatial characteristic of a given design element, such as the font weight and size, or the thickness of a line.

See FIGS. 10A, 10B and 10C for screenshots of a contrast calculator using the present invention. Notice how the example fonts change in size as the contrast value decreases from 75, down to 51, and finally down to 29, which is below the recommended cut off for text.

The specific exponents and other constants for scaling or offsets as needed for the algorithm, can be preset and stored in computer code, derived from the results of empirical studies, or can be set dynamically based on inputs relating to environmental factors such as from an ambient light sensor.

While a pair of inputs is described here in the example for clarity and simplicity, any reasonable number of color inputs can be accommodated. For instance, consider an interactive component such as a button which has text on top. We would want to test the text using a foreground color, the button using a first proximal background color, and the second, larger proximal background around the button using a third color. Further, we may wish to have a fourth color input for the RMS average of the entire screen and a fifth of the surround illuminance to provide a Tacit Adaptation Value.

The lightness contrast for any specific element must be calculated relative to one or more adjacent colors. In the case of the example button, the contrast between the color of the button and the text is calculated, and the contrast between the color of the button and its proximal background is calculated. The input for an RMS average of the entire screen provides an estimated adaptation field that then determines or adjusts the exponents used to transform ESL to a perceptual lightness in preparation for determining the lightness contrast.

### Measuring Supra-Threshold Contrast Perception

As a physical quantity in the real world, light can be measured such as the measure of luminance in cd/m². However, there is not a linear relationship between measured luminance and the perception of lightness, darkness, and brightness. Perceptions such as lightness happen as a context sensitive function of the human vision system, and as a result cannot be directly measured.

See FIG. 3B which is a graph of a number of human lightness perception curves, and the curve that is appropriate at any given moment is based on the context and current environmental conditions. FIG. 3A shows some of the perception curves in the present invention for its model of lightness contrast perception. Notice that the power curves are different based on the polarity of the stimulus. Notice also that the foreground and the background are using different exponents. Doing so allows for easy shaping of the contrast results to match empirical data.

To gather data, it is necessary to perform experimental tests with human observers under defined conditions, to determine the human perceptual response to a given range of stimuli, and from that we can then create a mathematical model that allows us to calculate reasonable predictions of how a given set of stimuli will be perceived.

For an example of such an experiment, consider the 1920s W. D. Wright and J. Guild color-matching experiments, which measured the boundaries of human color vision. In these experiments, a reference light with a spectrally pure color was presented to a human observer, adjacent to the reference light was light comprised of a mixture of a plurality of colored “primary” lights, each with adjustable intensities, nominally red, green, and blue. The human observer would then modify the intensity values of the adjustable colored lights until they created a visible match to the reference light. The numerical values of the adjustable colored light intensities were used to create a mathematical model of the gamut of human color perception, generally referred to as the “CIE 1931 Standard Observer”.

Earlier we established that contrast of text at the bare minimum threshold of legibility is insufficient for readability at best speed and comprehension. Therefore, we need to establish a measure of perceptual contrast related to a supra-threshold range, which we refer to as the readability contrast of text. The present invention describes a method and means for empirical study of human perception of contrast at supra-threshold levels, useful for specifying readability contrast.

The preferred embodiment of the invention as it pertains to collecting empirical data to form a model of human contrast perception comprises means to display to a study subject a plurality of patterns of high spatial frequency stimuli, such as but not Limited to text, where the colors for the stimuli pattern or the background or both, are adjustable by the study administrator, and selectively, the administrator can permit the study subject to adjust one or more colors as needed for the specific perception data to be collected.

As has been long-established, human visual perception does not have a linear relationship to external light stimuli, as perception is affected substantially by surrounding context, the current light adaptation of the eye, and the amount of change of, or a difference between, two stimuli. Further, this non-linear relationship changes in characteristics the farther the stimuli difference is from the threshold JND. As such, a linear extrapolation from a threshold value will not result in a perceptually uniform model. Instead, measurement needs to be conducted at a supra-threshold level.

One way to do this is by magnitude estimation, however, contrast perception is affected by psychophysical factors such as contrast constancy and contrast adaptation, both of which prevent accurate estimation of a given contrast magnitude without a defined reference to compare to.

The present invention solves this problem by presenting spatially identical sets of stimuli 204 onto a display screen, where there is a first color 202 that is common between two stimuli patterns, with a second color 208 for the first stimuli being darker, and a second color 212 for the second stimuli being lighter. With this configuration, the test study subject can adjust the common color until they find a point of visual equilibrium between the two contrasts, where each visual contrast is perceptually the same and/or is equally readable compared to the other.

This visual equilibrium point, defined by the luminance of the middle color, is the “center of contrast” between the darkest and the lightest colors. Once we know the center of contrast then we then have three points with which we can begin to estimate a curve that defines the nonlinear relationship of contrast between colors at various lumenances and various positions in the visual range.

We can then choose that center contrast point as a new high luminance versus the low color that was used in the previous trial, and find the center contrast between these two closer colors, and in so doing further define the curve of perception. Working through a range of colors this way we will find a collection of perception curves that are slightly different depending on polarity and the total contrast, and also the spatial characteristics of the stimuli and the larger surrounding backgrounds and the environmental illumination, and so forth.

Therefore, the present invention also includes an adjustable screen background color 220, so that the overall screen luminance can be changed to modulate the adaptation state of the test subject. Further, the test area has a controllable lighting system which can be adjusted for a specific level of ambient illumination, and materials of a specified reflectivity around the display screen to provide a stable surround luminance value.

### The Experimental Reference Setup Comprises:

The display device to be tested.

A suitable light meter to measure the ambient lighting.

A suitable device for measuring or calibrating the display device, such as a colorimeter or a spectrophotometer.

A desk for mounting the display device, and the experimental controls for the test subject to manipulate.

A stable chair for the test subject, which does not have wheels and does not recline to reduce but movement of the test subject during the trials.

A light-translucent material draped around the test area which can be illuminated from behind with adjustable intensity illumination, to provide a diffuse ambient illumination at a controllable, specific level, to create the adapting field.

A solid, opaque, and diffusely reflecting material surrounding the display device, to substantially fill the field of view of the test subject. Said material with a specific diffuse reflectance as needed for the given experiment, an example being the common 18% grey.

The background area of the screen of the display device can be adjusted by the test administrator, or set as a function of an automated test program, to any given level the display device is capable of, said background adjustment being completely independent of the stimulus or other specific experiment colors.

The test stimulus presented on the display device comprises:

At least three independent colors, a darkest color 208, a middle color 202, and a lightest color 212.

The test administrator or an automated test program can set at least two of the stimulus patterns colors, leaving at least one color for the test subject to adjust as a function of the experiment.

Depending on the specific experiment configuration, the colors may be assigned to a stimulus pattern such as text 204, or they may be assigned to the immediate proximal background 202 around the stimulus.

Displayed on the screen are at least two lines of stimulus patterns 204 such as text, each against a different background or each line of text being a different color, or some combination thereof.

The stimulus patterns use fonts of the size and weight or other stimuli at a size and thickness, as needed to measure for a given use case. For example, to determine the lightness contrast relative to body text, the stimuli pattern should be a block of body text at a nominal body text size.

Preferentially the stimulus patterns should include a range of font weights and or a line thickness, and arranged in such a way that they progress from thicker to thinner.

Preferentially, each instance of the stimulus pattern using the darker color can be lined up and adjacent to an identical stimulus pattern which is using the lighter color, so that the differences or similarities between the two stimulus patterns are easy to discern.

For experiments using the three-color configuration, there are four basic set ups where the test administrator or automated test program presets the darkest and the lightest color, and subsequently the test subject adjusts only the middle color. The four basic set ups all use at least two lines of identical text and/or spatially identical stimuli patterns are:

FIG. 5E the middle color is the text color for all lines of text, and the darkest and the lightest colors each provide the proximal background for at least one of the lines.

FIG. 5F the middle color is the proximal background behind all lines of text, and the darkest and the lightest colors each are assigned to at least one line of text.

FIG. 5G the middle color is the proximal background for a first line of text, where the first text is the darkest color, and the middle color is also the text color for a second line of text and the lightest color is the proximal background for the second line.

FIG. 5H the middle color is the proximal background for a first line of text, where the first text is the lightest color, and the middle color is also the text color for a second line of text and the darkest color is the proximal background for the second line.

A means is provided to record data regarding the testing conditions, the test subject's demographics, and the specific adjustments that the test subject made for each trial.

A means is provided to calculate the lightness contrast value from at least one set of collected study data, and where the lightness contrast values for the given luminance data are displayed 224, and where the calculation algorithm makes use of exponents for which means are provided to adjust them 226 228 230 232, and view the updated result as the adjustments are being made. This allows creating a set of exponents to use with the contrast calculation algorithm, and to easily adjust the exponents to find an equilibrium where the reported contrast value matches that of the perceptual study data, thereby supporting the creation of a perceptually uniform contrast calculation.

### Conducting the Study

The test administrator sets the ambient lighting, screen background color, and configures the colors of the stimuli patterns, either manually per trial or through the use of an automated test program.

The test subject is given instructions on how to adjust the middle color and how to judge when the middle color produces a matching contrast between the darker and the lighter color pattern.

For each trial set of lighter and darker colors, the test subject will adjust the middle color one or more times, and the test administrator or automated test program will record that color value each time the test subject completes an adjustment.

The recorded information for each trial includes the color as adjusted by the test subject, and the polarity against the darker and the lighter color, in other words, if the middle color was being used as the text color or as the proximal background color.

### Adjusting the Perceptual Contrast Model

The contrast algorithm uses independent exponents for each input color, where one input is designated for the text and another for the immediate proximal background, and in some embodiments, additional inputs for an interactive control, surrounding background field, dynamic adapting field, or other light or color field for the specific application of use. These exponents are preferentially predetermined and specific to a polarity, in other words which color is lightest and which one is darkest. For use cases where an adapting field is used, the adapting field provides a luminance value that is used to adjust the exponents for in the contrast calculation.

The contrast algorithm provides means to use scale factors and offsets based on polarity, and the assumed or measured adapting field, in order to maintain the results within a desired range, and to adjust for perceptual uniformity.

To determine the values of the exponents and other constants, a computer program is employed, where the recorded luminance data from the experimental study is loaded into memory, and interactive adjustment tools are provided to allow adjusting the exponents and other constants, while viewing the resulting lightness contrast values, calculated from the experimental study data and using the adjusted exponents and constants. See FIG. 4 for an example of the console used for adjusting the various exponents in real time to fit the model to the empirical data collected. The sliders on the upper level 200 206 210 adjust the experimental intensity values of the different colors. The sliders and numerical entry boxes 234 through the middle section directly affect the exponents and other constants used for scaling or for offsetting results.

The very long slider 236 which is set at 2.4 in this image is the exponent used for linearizing the input color primary values. The four smaller sliders 226 228 230 232 immediately above the long slider are the individual sliders for the exponents for the text and the proximal background inputs. Each of those small pairs of sliders can be locked together so that moving one moves the other to the same degree. They can also be selectively locked in opposition, so moving one in one direction will cause the other to move in the opposite direction to the same degree. The large square panel 224 in the lower left provides calculations of contrast that update in real time as the sliders are manipulated. It is advantageous for perceptual uniformity to converge the ratios to 1:1; this is not a contrast measure itself; this is the ratio of contrast against another contrast, indicating that there is a balance between the upper and the lower half of the contrast range under test.

Because the experimental study is built on the concept of finding the middle contrast between two colors, this implies that the contrast value between the middle color and the darkest color or the middle color and the lightest color, is half that of the contrast between the darkest and the lightest color without regard to the middle color. It is also implied that with perceptual uniformity the contrast value between the middle color and the darkest color should be roughly equal to the contrast value between the middle color and the lightest color, for each polarity. It is expected that each polarity may exhibit a different middle contrast value, as positive and negative polarities track differently across most of the visual range.

Therefore, the exponents and scale factors are adjusted such that the contrast middle represents half of the contrast between any given darker or lighter color. As we approach very low or very high contrasts, additional functions are required to maintain perceptual uniformity, due to differences that occur at threshold levels versus supra-threshold.

Preferentially, the exponent for the darker color in each polarity is slightly larger than the exponent for the lighter color, this prevents undesired crossover or other anomalies at very low luminance values. Experimental data may also indicate using different linearization exponents and coefficients in order to emulate the output of physical monitors, or to pre-shape luminance data.

### Determine Polarity and Compensate for Protanopia

In the preferred embodiment and in conjunction with the estimated screen luminance calculation, an additional function adjusts the values of the red primary's intensity, to effectively reject or limit colors involving saturated reds or oranges when those colors are paired with a darker color. This helps accommodate protanopia, which is a form of color vision deficiency that is insensitive to red.

Means are provided to perform a multi-step method to compensate for protanopia, where:

There is a defined color space with a plurality of color primaries, nominally Red, Green, and Blue.

There is a foreground (text) color A and a proximal background color B to be tested, both of which are referenced to the defined color space.

There is a pre-calculated protan luminance factor (PLF) which specifies the amount of luminance reduction of the red color primary when viewed by protanopia as opposed to viewing by standard vision.

The values of each of the Red, Green, and Blue primaries of both color A and B are linearized by applying the color space gamma independently to each.

The R, G, B spectral weighting coefficients are then applied to each corresponding primary value by multiplication.

The linearized, weighted values of Red A and Red B are temporarily stored in memory.

The linearized, weighted values of Red, Green, and Blue of color A are summed to make ESL A, and likewise for color B to make ESL B.

Determine if ESL A or ESL B is greater, and if Red A or Red B is greater, then:
IF ESL A is greater than ESL B AND Red A is greater than Red B, THEN
subtract red B from red A,
multiply the result by PLF to make OFFSET A,
subtract OFFSET A from ESL A
IF ESL A is now less than ESL B, THEN return 0,
ELSE calculate for reverse (negative) polarity contrast.
ELSE IF ESL B is greater than ESL A AND Red B is greater than Red A, THEN
subtract red A from red B,
multiply the result by PLF to make OFFSET B,
subtract OFFSET B from ESL B
IF ESL B is now less than ESL A, THEN return 0,
ELSE calculate for normal (positive) polarity contrast.

### Calculating Lightness Contrast

Each individual ESL used in the contrast calculation must have an exponent applied so that the ESL is following an appropriate power curve relative to perception, as needed for the contrast calculation. In the preferred embodiment these exponents are predetermined utilizing the previously described empirical data from contrast matching experiments, and aligning the exponents to create a model that fits the experimental data.

Steps for calculating lightness contrast comprise:

After the ESL is determined for each color, pre-process each ESL to soft clamp very low luminance values to compensate for screen flare and the common nonlinearities or noise that occur near black.

The soft clamp for the preferred embodiment includes a clamp exponent and a clamp threshold.
IF the ESL value is less than the clamp threshold THEN
subtract the ESL value from the clamp threshold,
apply the clamp exponent to that result,
add that result back to the ESL, and continue.

For positive polarity use the exponent set and constants defined for positive, and likewise if the polarity is negative use the negative polarity exponent and constants set.

Apply the text exponent for the polarity to ESL A to create TextLightness.

Apply the background exponent for the polarity to ESL B to create BGLightness.

Subtract TextLightness from BGLightness for the raw S_{apc} value.

To fine-tune the Lc value output, and adjust perceptual uniformity, apply the following when appropriate:
IF the absolute value of S_{apc} is less than a predetermined output clamp threshold, return 0,
ELSE multiply the result by the predetermined scale factor and apply the predetermined offset.

The result of these calculations is a perceptually uniform lightness contrast value, preferentially designated L^{c}. For sRGB web content, the draft values in use at the time of this application:

The range for light mode is L^{c} 0 to 106.

The range for dark mode is L^{c} 0 to −108.

Light mode provides a positive value, and dark mode provides a negative value, however the perceived contrast is the distance from zero, so dark mode L^{c}−60 is perceptually similar to light mode L^{c} 60,

Or put another way, Lc value indicates whether or not the text or other small design element such as an icon, should be brighter or darker than the background. With a negative value, the background should be darker. However, the absolute value of the lightness contrast determines the design guidance in terms of the spatial aspects such as font weight and size.

### Utilizing Lightness Contrast

While integrating perceptually uniform lightness contrast as recited in this disclosure may be more complicated than some of the simpler legacy methods, the substantially greater accuracy afforded by perceptual uniformity provides for more flexible design guidelines, and enhanced readability contrast.

### Dark Mode and More

Being perceptually uniform, the L^{c} value can be used in conjunction with automated color selection functions assuring that a given level of contrast is maintained. The perceptually uniform L^{c} value is particularly useful for automatically inverting a color scheme, where a light mode color scheme is inverted into a dark mode color scheme, maintaining the same contrast relationships between elements.

Other modes such as enhanced contrast, reduced contrast, and enhancements for various color vision related issues are all facilitated by this perceptually uniform method.

### Readability Contrast Guidelines

The L^{c} value as it relates to a given level of readability, and the associated font size and weight recommendations, can be determined in relationship to the critical size and critical contrast levels as discussed in the seminal readability research of Drs. Steve Whittaker, Ian Bailey, and Jan Lovie-Kitchin et alia.

In one embodiment, means are provided to use the L^{c} value in conjunction with an interpolatable lookup table, to provide recommendations for font size and weight based on the calculated contrast. The advantage of an interpolatable lookup table is that it provides a simple way to conform to an alternate language or writing system by simply substituting a table developed for the desired writing system. L^{c} value can be used to provide guidance regarding spatial characteristics of design elements such as the size and weight of a font or the thickness of a line, in accordance with the empirical peer-reviewed readability research mentioned above.

### Basic Spatial Algorithm

A second embodiment uses a more direct, algorithmic approach. Instead of a lookup table for a given writing system, the algorithm provides mathematical functions which directly translate a L^{c} contrast value to a specific spatial quality of a design element, such as font weight or size, or line thickness or element size.

This example we will use the CSS reference px, which is a unit of size used in web-based content, and it is defined as 1.278 arc minutes of visual angle subtended onto the retina. For our example of an sRGB display, divide the L^{c} value by 100, and then raise it to the −1.5 power to find the recommended line thickness in px. Further multiply that value by 6 to find the recommended font's x-height for a normal weight (400) Font, divide the x-height by 0.52 to find the reference Font size.

lineThickness=(LcValue/100)^{-1.5}

xheight=lineThickness*6

fontSize=xheight/0.52

For this example, start with lightness contrast L^{c}60. Divide by 100 for 0.6 and then raise that to the −1.5 power to get a recommended line thickness of 2.1 px, multiply by 6 and then divide by 0.52 for a suggested font size of 24 px. These are example values, different goals for readability and discernibility, as well as different color spaces and environments may result in different values for a given application.

In the creation of tools for designers and developers, it is advantageous to be able to choose one color and a contrast and have the system do a reverse calculation to find the other color. That is possible with the present invention, and a reverse function is part of the standard library of computer code of the main embodiment.

### Flexible and Versatile

The used case and application for the present invention is not limited to design guidelines, through our testing and research we found that there is significant potential for utilizing the present invention for medical and clinical use. For instance, the study experiment can also provide insight into contrast deficiencies experienced by some test subjects, indicating a potential for clinical use, especially for profiling certain aspects of visual function in a dynamic way.

Because this technology is easily integrated into dynamic environments, use in embedded systems is anticipated, in aerospace, on flight decks, transportation, automotive, and certainly military uses. Equipped with an ambient light sensor, the present invention can provide rapid and useful adjustments for use with self-illuminated displays or for automatically controlling certain types of lighting systems.

The present invention's unique capabilities relating to perceptual uniformity, make it functional over a wider range of ambient illumination conditions than many previous technologies which are currently in use today. In other words, it responds on a dynamic basis to changing conditions in a manner that is useful for human occupied environments.

The above specification and descriptions are intended to be illustrative and not restrictive. Although the disclosure described herein presents practical embodiments, it is apparent to those skilled in the art that the present invention may be practiced in embodiments without certain specific elements, and that the algorithms, systems, and methods presented herein are not inherently related to any particular computer or other apparatus.

A variety of general-purpose systems may be used with methods and algorithms as disclosed herein, or it may prove useful to construct a more specialized apparatus to perform the required method steps, which may be used without departing from the spirit and scope of the invention. In the description, well-known components or methods are not described in detail in order to avoid unnecessarily obfuscating the present invention.

Further, the description recites an abundance of specific details including examples of specific systems, components, methods, to facilitate a useful understanding of the various embodiments of the present invention. The present invention is not restricted to the particular constructions described and illustrated herein.

1) An apparatus to quantify supra-threshold visual contrast perception, comprising:
a means to display a plurality of visual stimuli patterns for viewing by a test subject; further comprising:
a plurality of foreground patterns overlaid onto one or more background elements;
a means to adjust and manipulate the visual presentation properties of the stimuli, selectively as groups of stimuli or as independent stimuli;
a means to adjust the overall display background, and the ambient light conditions of the test environment; a first instance of a specific pattern of stimuli, visually displayed with a first foreground color and overlaid onto a first background color;
a second instance of the specific pattern of stimuli, visually displayed with a second foreground color and overlaid onto a second background color;
a means to set each of the first and second foreground and background colors to specific values; a variable control means to adjust one or more specific colors over a continuous range while simultaneously viewing the first instance and the second instance of the stimuli, and with numerical values hidden from the view of the test subject;
a means to store the numerical values of each color as set or adjusted for later retrieval and study.

2) The apparatus of claim 1 further comprising a means to selectively link colors together so that they are identical, comprising at least these selection choices:
A) the first foreground color to the second foreground color;
B) the first background color to the second background color;
C) the first foreground color to the second background color;
D) the first background color to the second foreground color;
a means to associate the linked color to the variable control means so that the linked color can be adjusted by the test subject.

3) The apparatus of claim 2 further comprising a means to limit the minimum value and maximum value of the variable control means and the associated linked color, where:
the minimum value of the linked color is no darker than the darkest unlinked stimulus color;
the maximum value of the linked color is no lighter than the lightest unlinked stimulus color.

4) The apparatus of claim 2 wherein the first instance and the second instance are displayed adjacent to each other, and aligned such that each element of the pattern of the first instance is adjacent to the matching element in the pattern of the second instance.

5) The apparatus of claim 2 further comprising three or more instances of the patterns of stimuli, and a means to create two or more linked colors, with each linked color associated with an independent variable control means.

6) The apparatus of claim 2 further comprising one or more processors; and a memory in communication with the one or more processors, the memory comprising executable instructions that, when executed by the one or more processors, cause the device to perform the functions of claim two, and additionally provides a means to selectively save in memory the numerical values of each color, and the specific element that color was associated with.

7) The apparatus of claim 6 further comprising means to calculate lightness contrast of at least one set of stimuli data as adjusted by the test subject, and where means are provided to experimentally adjust the values of the independent exponents applied to each of the estimated screen luminances, while viewing a display of the contrast values being calculated, such that exponents can be derived which match the stimuli data in a perceptually uniform way.

8) An algorithm to calculate a perceived visual contrast of a foreground color against an adjacent proximal background color, comprising:
a predetermined first exponent and a second exponent to be used with a positive polarity condition;
a predetermined third exponent and a fourth exponent to be used with a negative polarity condition;
a first luminance value derived from the foreground color;
a second luminance value derived from the adjacent proximal background color;
an output contrast value;
and further comprising the following steps:
A) determine if the first luminance value is lower than the second luminance value; then:
B1p) if the first luminance value is lower, it is the positive polarity condition;
then calculate a first lightness curve by raising the first luminance to the power of the first exponent;
calculate a second lightness curve by raising the second luminance to the power of the second exponent;
B2n) else, if the first luminance value is not lower, it is the negative polarity condition;
then calculate the first lightness curve by raising the first luminance to the power of the third exponent;
calculate the second lightness curve by raising the second luminance to the power of the fourth exponent;
C) finally, calculate the perceived visual contrast by subtracting the first lightness from the second lightness;
the output contrast value is the result of step C.

9) The algorithm of claim 8, further comprising:
a third luminance value derived from an encompassing background color that surrounds the adjacent proximal background associated with the second luminance;
and a means to adjust the first, second, third, and fourth exponent values, in accordance with the expected change in perception and adaptation state in relation to the luminance of the encompassing background color.

10) The algorithm of claim 8; further comprising:
a transform method to convert a numerical color value to an estimated screen luminance, to be applied prior to step A of claim eight, where:
the color value is referenced to a defined color space representing a physical display comprising a plurality of color primaries;
the color value contains a numerical primary value for each color primary of the defined color space;
the transform method utilizes a linearizing exponent determined by the physical display's EOTF characteristics, and a set of predefined coefficients containing a specific coefficient assigned to each primary of the defined color space;
the transform method proceeds with the following steps:
A) normalize the numerical primary values such that each primary value is relative to 0.0 for a black reference and 1.0 for a white reference;
B) create a set of linearized primary values by raising each normalized primary to the power of the linearizing exponent;
C) create a set of weighted primary values by multiplying each linearized primary by the specific coefficient for that primary;
D) sum the weighted primaries from step C together to create the estimated screen luminance;
E) the process continues with step A of claim eight, where:
the first luminance value is the estimated screen luminance of the foreground color;
and the second luminance value is the estimated screen luminance of the adjacent background color.

11) The algorithm of claim 10, further comprising:
a means to adjust the coefficient of a specific primary of the defined color space to generate an alternate estimated screen luminance to provide enhanced accommodation for certain user needs; where:
a predetermined offset factor to reduce the specific primary's contribution to luminance and therefore contrast in certain situations;
comprising the following steps:
A) process thru step C of claim ten and stop;
B) temporarily store in memory the weighted primary values of the specific primary for the first luminance and the second luminance as first primary and second primary;
C) continue processing at step D of claim ten, thru step A of claim eight and stop; D1p) if it is the positive polarity condition AND second primary is greater than first primary, then subtract the first primary value from second primary, and apply the predetermined offset factor to the result, then subtract that result from the second luminance, and continue processing with step B1p of claim eight;
D2n) if it is the negative polarity condition AND first primary is greater than second primary, then subtract the second primary value from first primary, and apply the predetermined offset factor to the result, then subtract that result from the first luminance, and continue processing with step B2n of claim eight.

12) The algorithm of claim 10, further comprising: a user interface means to enter a plurality of color values, where are each color input is clearly marked as to the purpose of that color and which foreground or background element it is to be assigned to;
a display means to present a plurality of samples of spatially-ordered sample elements, which are displayed using the colors as entered into the color inputs, and which adjust and update their spatial characteristics based on the calculated lightness contrast as the colors are entered or adjusted.

13) The algorithm of claim 12, further comprising:
a user interface means to selectively choose the specific plurality of spatially-ordered sample elements from an available plurality of sample elements;
where the sample elements include a variety of useful examples for the specific design objectives.

14) The algorithm of claim 8, further comprising: a soft-clamp to apply to the first and second luminance values if either is below a threshold value, by incrementally adding a positive offset, comprising:
the black threshold value which defines the point to begin adding a scaled amount of positive offset to the luminance value;
a black clamp value, which determines the maximum positive offset when the said luminance value is at zero;
a means to incrementally add the positive offset to said luminance value below the black threshold value, with the maximum offset to add being determined by the black clamp value;
and the soft-clamp to be applied immediately prior to step A of claim eight.

15) The algorithm of claim 14, further comprising: said first, second, third, and fourth exponent values adjusted to optimize the results for a desired perceptual uniformity of stimuli of a desired spatial frequency, as displayed on a reference display in a reference environment, wherein:
said exponent values are individually adjusted such that a desired range of difference values of the first and second lightness curves for both the positive and negative polarity conditions fit within an acceptable deviation range, relative to a data set aggregated from empirical studies;
said data set providing adequate statistics to support the desired ranges, polarity conditions, and spatial frequencies;
and preferentially ensuring that: the first exponent is greater than the second exponent; the fourth exponent is greater than the third exponent.

16) The algorithm of claim 14, further comprising:
a means to scale the output result such that the desired perceptual uniformity is enhanced and said output contrast value is within a desired range;
a means to clip the output contrast value to zero for all values below a minimum output contrast value threshold;
said minimum output contrast value threshold is no less than the lowest 10% of the output contrast value range.

17) The algorithm of claim 14, further comprising:
a means to scale the output result such that the desired perceptual uniformity is enhanced and said output contrast value is within a desired range;
a means to create an extended low range contrast value that extends down to two percent or less of the total output contrast value range;
a means to prevent incorrect polarity reversals in the extended low range contrast value.

18) the algorithm of claim 17, for the comprising:
a means to calibrate the extended low range contract value to a given clinical contrast sensitivity standard;
a means to display two or more identical stimuli patterns of a defined spatial frequency, and with adjustable colors, and overlaid onto one or more backgrounds with adjustable colors;
a means for a user to adjust the stimuli colors while simultaneously viewing the stimuli patterns;
a means to selectively store the color values as adjusted by the user for later retrieval and study.

19) A method of predicting visual contrast in a perceptually uniform way, capable of useful contrast calculations for light-mode, dark-mode, other enhanced modes, and providing improved readability and visual accessibility, comprising the steps of:
A) select a first color for text and a second color for the adjacent proximal background;
B) convert the first and second colors to estimated screen luminance by normalizing the primary values to a defined range; then
C) linearize the first and second color primary values by applying a predefined gamma or tone response curve; then
D) multiply each linear primary value with its predefined coefficient; then
E) separately sum the results of the weighted primaries for each color, creating a first ESL and a second ESL;
F) calculate contrast by subtracting the first ESL from the second ESL;
G) then apply the scale and offset for the resulting L^{c} contrast value.

20) The method recited in claim 19, further comprising the steps:
A) process claim nineteen through step D, then
B) temporarily hold the weighted primary values of the primary-to-be-offset from the first luminance and from the second luminance, as first primary and second primary;
C) continue processing step E of claim nineteen and stop;
D1p) if the first ESL is less than the second ESL AND first primary is less than second primary, then subtract the first primary value from second primary, and apply the predetermined offset to the result, then subtract that result from the second luminance, and continue processing with step F of claim nineteen;
D2n) if the first ESL is greater than the second ESL AND first primary is greater than second primary, then subtract the second primary value from first primary, and apply the predetermined offset to the result, then subtract that result from the first luminance, and continue processing with step F of claim nineteen.
